(19) United States
(12) Patent Application Publication
(10) Pub. No.: US 2024/0117307 A1
(43) Pub. Date: Apr. 11, 2024

(54) THYMIC CELL COMPOSITIONS AND METHODS OF USE THEREOF

(71) Applicant: Thymmune Therapeutics, Inc., Cambridge, MA (US)

(72) Inventors: Bing Lim, Cambridge, MA (US); Edroaldo Lummertz da Rocha, Cambridge, MA (US)

(21) Appl. No.: 18/572,159

(22) PCT Filed: Jun. 22, 2022
(86) PCT No.: PCT/US2022/034579

§ 371 Date: Dec. 19, 2023

Related U.S. Application Data
(60) Provisional application No. 63/321,142, filed on Mar. 18, 2022.
(60) Provisional application No. 63/296,250, filed on Jan. 4, 2022.
(60) Provisional application No. 63/256,445, filed on Oct. 15, 2021.
(60) Provisional application No. 63/214,176, filed on Jun. 23, 2021.

(51) Int. Cl. C12N 5/078; C12Q 1/6881

(52) U.S. Cl. CPC C12N 5/0634; C12Q 1/6881; C12N 2506/45

(57) ABSTRACT

The present disclosure provides methods for generating and maintaining thymic cell in vitro. Compositions and systems of cell populations that include thymic cells are also provided herein. In one aspect, the disclosure provides a method for generating a population of thymic cells in vitro by culturing a population of cells in the presence of a soluble factor, a mineral or a combination thereof to induce differentiation or maturation of the population of cells to thymic cells; wherein the population of cells is optionally engineered to express a cell surface receptor or an intracellular factor; and wherein the population of cells comprises one or more cell types selected from the group consisting of pluripotent stem cells (PSCs), definitive endodermal (DE) cells, third pharyngeal pouch endodermal (PPE) cells, and anterior foregut endodermal (AFE) cells.

### CROSS REFERENCE TO RELATED APPLICATIONS

This application claims benefit of priority under 35 U.S.C. 119(e) to U.S. Provisional Application No. 63/214,176 filed Jun. 23, 2021, U.S. Provisional Application No. 63/256,445 filed Oct. 15, 2021, U.S. Provisional Application No. 63/296,250 filed Jan. 4, 2022, and U.S. Provisional Application No. 63/321,142 filed on Mar. 18, 2022, the contents of which are herein incorporated by reference in their entirety.

### BACKGROUND INFORMATION

The thymus is a primary lymphoid organ that plays a central role in the immune system. The microenvironment of the thymus provides a unique training ground for the development of maturation of effector cells such as lymphocytes (e.g., T cells). It is well known in the art that the complex interactions between thymic cells and effector cells can determine the phenotype and functionality of the effector cells. Some thymic cell-effector cell interactions are tuned such that recognition of factors expressed by the thymic cells promotes the survival of the effector cells. In contrast, other thymic cell-effector cell interactions may result in the death of the effector cells. By controlling such interactions, the thymus plays a pivotal role in establishing a repertoire of effector cells that are able to mount an activated immune response to foreign invaders while establishing tolerance to self.

There remains a need for improved methods of generating thymic cells, thymic cell products, and for cell populations enriched in functional thymic cells that can differentiate into functional thymic epithelial cells.

### SUMMARY

The present disclosure provides thymic cell products and methods of making thereof. The thymic products of the disclosure are prepared by (i) generating one or more population of thymic cells; (ii) extracting and sequencing RNA from each of the population of thymic cells; (iii) analyzing the RNA sequences to identify sub-populations within each population of thymic cells to generate a test thymic map for each of the population of thymic cells; (iv) comparing each of the test thymic maps to a reference thymic map; and (v) identifying a thymic cell product by selecting a test thymic map that has a Sub-Population Ratio (SPR) or a Relative cTEC Ratio (RCTR) of from about 0.7 to about 1.4. In some aspects, the sub-populations of cells within each of the thymic cell populations are selected from immature thymic epithelial cells (iTECs), cTEC-high cells, cTEC-low cells, Aire+ mTEC-high cells, mTEC-low cells, Corneocyte like mTEC cells, Ciliated cells, Myelin cells, Myoid cells, Neuroendocrine cells and/or Tuft/lonocyte cells. In some aspects, the SPR is from about 0.7 to 1.4. In some aspects, the SPR is a iTEC SPR, a cTEC-high SPR, a cTEC-low SPR, a Aire+mTEC-high SPR, a mTEC-low SPR, a Corneocyte like mTEC SPR, a Ciliated SPR, a Myelin SPR, a Myoid SPR, a Neuroendocrine SPR and/or a Tuft/lonocyte SPR. In some embodiments, the SPR is an iTEC SPR. As a non-limiting example, the iTEC SPR is 1.

In some embodiments, the RCTR is from about 0.7 to 1.14. As a non-limiting example, the RCTR is 1.

The thymic cell populations of the disclosure utilized for preparing the thymic cell product is prepared by the differentiation of stem cells.

In some embodiments, the reference thymus map is generated using human thymi e.g. adult or fetal thymi.

In some embodiments, the iTEC subpopulation of the population of thymic cells may include expression of one or more of IGKC (ENSG00000211592), ANAX2, LIMA1 (ENSG00000050405), EGFR (ENSG00000146648), ASCL1 (ENSG00000139352), HES1 (ENSG00000114315), JUND (ENSG00000130522), FOS (ENSG00000170345), ARID5B (ENSG00000150347), IRF1 (ENSG00000125347), MAFB (ENSG00000204103), IFI16 (ENSG00000163565), FOXC1 (ENSG00000054598), STAT1 (ENSG00000115415), JUNB (ENSG00000171223), EGFR1, ZFP36 (ENSG00000128016), JUN (ENSG00000177606), FOSB (ENSG00000125740), IER2 (ENSG00000160888), PAX9 (ENSG00000198807), and/or HIF1A (ENSG00000100644).

In some embodiments, the cTEC subpopulation of the population of thymic cells may include expression of one or more of PSMA3 (ENSG00000100567), FABP5 (ENSG00000164687), APRT (ENSG00000198931), LSM6 (ENSG00000164167), CTSV (ENSG00000136943), SNRPE (ENSG00000182004), ECHS1 (ENSG00000127884), HSPE1 (ENSG00000115541), RAN (ENSG00000132341), TMA7 (ENSG00000232112), TIMM13 (ENSG00000099800), LDHB (ENSG00000111716), ECI1 (ENSG00000167969), GCSH (ENSG00000140905), NOP58 (ENSG00000055044), MRPL11 (ENSG00000174547), STOML2 (ENSG00000165283), ING2 (ENSG00000168556), TOMM7 (ENSG00000196683), MRPS34 (ENSG00000074071), MRPL14 (ENSG00000180992), MRPL57 (ENSG00000173141), IMP3 (ENSG00000177971), MZT2A (ENSG00000173272), and XRCC6 (ENSG00000196419).

In some embodiments, the corneocyte-like mTEC subpopulation of the population of thymic cells may include expression of one or more CD24 (ENSG00000272398), ELF3 (ENSG00000163435), CLDN4 (ENSG00000189143), MAL2 (ENSG00000147676), ASAH1 (ENSG00000104763), TMEM123 (ENSG00000152558), TMBIM6 (ENSG00000139644), LGALS3 (ENSG00000131981-), MYL12B (ENSG00000118680), ACADVL (ENSG00000072778), KRT19 (ENSG00000171345), SAT1 (ENSG00000130066), RAB25 (ENSG00000132698), WFDC2 (ENSG00000101443), VAMP8 (ENSG00000118640), SPINT1 (ENSG00000166145), SERPINB1 (ENSG00000021355), CDH1 (ENSG00000039068), GSN (ENSG00000148180), SDC4 (ENSG00000124145), MGST2 (ENSG00000085871), CAST (ENSG00000153113), B4GALT1 (ENSG00000086062), PERP (ENSG00000112378), and DMKN (ENSG00000161249).

The present disclosure provides methods for generating thymic cells in vitro. These methods may include culturing a population of cells in the presence of a soluble factor, a mineral or a combination thereof. Such methods may induce differentiation or maturation of the population of cells to thymic cells. The population of cells may also be engineered to express a cell surface receptor or an intracellular factor. In some embodiments, the population of cells may be pluripotent stem cells (PSCs), definitive endodermal (DE) cells, third pharyngeal pouch endodermal (PPE) cells, anterior foregut endodermal (AFE) cells.

The thymic cells are one or more of immature thymic epithelial cells (iTECs), cTEC-high cells, cTEC-low cells, Aire+mTEC-high cells, mTEC-low cells, Corneocyte like mTEC cells, Ciliated cells, Myelin cells, Myoid cells, Neuroendocrine cells and Tuft/lonocyte cells, or combinations thereof. Soluble factors include, but are not limited to, growth factors, cytokines, transporters, hormones or combinations thereof. As a non-limiting example, the soluble factor is a growth factor such as epidermal growth factor (EGF; ENSG00000138798). The soluble factor is a cytokine e.g., macrophage inhibitory factor (MIF; ENSG00000240972). The soluble factor may also be a transporter e.g., Lactotransferrin (LTF; ENSG00000012223).

In some embodiments, the cells of the disclosure is cultured in the presence of a mineral. The mineral is iron, magnesium, calcium, manganese, molybdenum, phosphorus, potassium, sodium, sulfur, zinc, chloride, chromium, copper, fluoride or iodine. In one aspect, the mineral is iron. The cells of the disclosure is engineered to express a cell surface receptor or an intracellular factor for the purpose of generating or maintaining thymic cells. The cell surface receptor is CD74 (ENSG00000019582), integrin beta 1 (ITGB1; ENSG00000150093) and/or epidermal growth factor receptor (EGFR; ENSG00000146648). The intracellular factor is ENO1 (ENSG00000074800).

The present disclosure also provides methods of maintaining a population of thymic cells in vitro. The methods may include (i) culturing the population of thymic cells in the presence of a soluble factor or a mineral; (ii) culturing the population of thymic cells in the presence of one or more supporting cells; and/or (iii) engineering the population of thymic cells to express a cell surface receptor or an intracellular factor.

The cells of the disclosure is cultured in the presence of one or more supporting cells. Supporting cells are endothelial cells, mesenchymal stem cells, macrophages, dendritic cells (DCs), epithelial cells, fibroblasts, stromal cells, adipocytes, fibroblasts, vascular smooth muscle cells (VSMCs), or lymphatic endothelial cells. In some embodiments, supporting cells are mesenchymal stem cells. In some aspects, the supporting cells are endothelial cells.

### DETAILED DESCRIPTION

### I. Introduction

Thymic epithelial cells are important in T cell differentiation. Thymic cells prepared as described herein may permit exploitation of thymus tissue and thymus cell properties, e.g., thymus-related immune functions, for therapeutic applications. For example, it is known that age-related decline in immune function is caused by changes in the composition and functional capabilities of thymic cells. In addition, changes in sex hormones, including androgens and estrogens, cause the thymus itself to atrophy or become senescent. The onset of thymic atrophy may begin as early as the onset of puberty. Thus, regeneration of thymic epithelial cells may provide for compositions and methods that mitigate age-related decline in immune function.

### II. Compositions

### Cells

Cells of the disclosure may include, without limitation, thymic cells, effector cells, pluripotent stem cells, populations thereof and cells derived therefrom. In some embodiments, cells of the disclosure may be derived from pluripotent stem cells. In some embodiments, the thymic cells may be derived from induced pluripotent stem cells.

In some embodiments, cells of the present disclosure may be autologous, allogeneic, syngeneic or xenogeneic in relation to a particular individual or subject. In some embodiments, the thymic cells may be autologous, allogeneic, syngeneic, or xenogeneic in relation to subjects ultimately benefiting from their clinical application. In some embodiments, the cells of the disclosure may be mammalian cells, particularly, human cells. Cells may be primary cells or immortalized cell lines. In some aspects, the cells of the disclosure are prepared or derived from syngeneic cell sources. Any of the cells described herein are characterized by markers known in the art for that cell type.

### Thymic Cells

Cells of the disclosure may include a population of thymic cells. A thymic cell is a cell with one or more phenotypic or genotypic markers associated with a cell derived from the thymus or a cell destined to become a cell of the thymus. In some embodiments, the population of thymic cells are derived by the differentiation of pluripotent stem cells. In some embodiments, the pluripotent stem cells are iPSCs. The thymic cells are an embryonic, a fetal or an adult thymic cells. In some embodiments, the compositions of the disclosure may be populations of thymic cells. In some aspects, the compositions of the disclosure are thymic cell products.

In some embodiments, thymic cells are prepared from the differentiation of pluripotent stem cells that differentiate into thymic stems via one or more of the following steps: PSCs can differentiate and/or can be induced to differentiate into cells resembling the definitive endoderm (DE). Definitive endoderm cells can differentiate and/or can be induced to differentiate into cells resembling the third pharyngeal pouch endoderm (PPE). Definitive endoderm cells and/or PPE cells can differentiate and/or be induced to differentiate into cells resembling the anterior foregut endoderm (AFE). AFE can differentiate and/or be induced to differentiate into cells resembling the third pharyngeal pouch endoderm (PPE). Thymic epithelial progenitor cells (TEPCs) can be generated from PPE cells. TECs can be derived from TEPCS. Each of the cell types described herein are characterized by one or more markers. In some embodiments, pluripotent stem cells are associated with the increased expression of markers such as, but not limited to, OCT 4, SOX 2, and/or Nanog. In some embodiments, definitive endodermal cells are associated with the increased expression of markers such as, but not limited to, SOX17, FOXA2, CXCR4, and/or CER1. In some embodiments, anterior foregut cells (AFE) is associated with the increased expression of markers such as, but not limited to, FOXA2, SOX2, and/or PAX9. In some embodiments, the third pharyngeal pouch endodermal cells are associated with the increased expression of markers such as, but not limited to, HOXA3, TBX1, PAX9, EYA1, SIX1, PBX1, and/or PAX1. In some embodiments, thymic epithelial progenitor cells are associated with the increased expression of markers such as, but not limited to FOXN1, K5, K8, and/or HOXA3. In some embodiments, the thymic cells are derived from a DE cell, a third PPE cell, an AFE cell, a TEPC, and/or a TEC cell.

For the preparation of the population of thymic cells of the disclosure, pluripotent stem cells are cultured and differentiated to definitive endoderm cells. The definitive endoderm cells are further cultured and differentiated into anterior foregut cells. In some embodiments, the anterior foregut cells are cultured and differentiated into pharyngeal endoderm cells. In some embodiments, the pharyngeal endoderm cells are cultured and differentiated into, thymic cells, e.g., thymic epithelial cells. In some embodiments, the differentiation is performed from about 14 days to about 21 days.

In some embodiments, thymic cells are prepared from PSCs. In this regard, the methods may comprise culturing the pluripotent stem cells for a period of time and under conditions sufficient to differentiate the pluripotent stem cells into thymic cells. For example, the method may comprise culturing the pluripotent stem cells in the presence of the factors and/or inhibitors that drive the differentiation of PSCs to thymic cells.

In some embodiments, the methods for differentiating PSCs into thymic cells are any methods known in the art. The methods for differentiating PSCs into thymic cells may include the use of one or more parameters know in the art for differentiation or combinations thereof. The parameters include, but are not limited to, (i) factors promoting differentiation (ii) inhibitors promoting differentiation (iii) duration of time for promoting differentiation (iv) temperature (v) substrate and/or (vi) supporting cells that promote differentiation. Any of the methods or parameters for differentiating PSCs into thymic cells described in following references are used herein and include Parent et al. Cell Stem Cell. 2013 Aug. 1; 13(2):219-29; Soh et al. Stem Cell Rep. 2014 Vol. 2 j 925-937; Sun et al. Cell Stem Cell. 2013 Aug. 1; 13(2):230-6; Okabe et al. Cell. Reprog. 2015 Vol 17, No. 5; Su et al. Sci. Rep. 2015 5, 9882; Otsuka et al. Sci Rep 2020 10:224; International Patent Publications, WO2019060336, WO2020205859, WO2020220040, WO2014134213, WO2010143529, WO2011139628; WO2022076751, WO2014134213, WO2021222297 and Chinese Patent Publication CN201110121243; the contents of each of which are herein incorporated by reference in their entirety.

In some embodiments, the population of thymic cells may include sub populations of one or more cell types. The cell types are iTEC, mTECs, Corneocyte-like mTEC, cTEC-high, cTEC-low, mTEC-low cell.

In some embodiments, the population of thymic cells may include thymic epithelial cells (TECs). Thymic cells are or may be derived from TECs. In some embodiments, the TECs are derived by the differentiation of iPSCs. During embryonic development TECs are derived from non-hematopoietic cells which are negative for CD45 expression and positive for epithelial marker EpCAM. TECs are cortical thymic epithelial cells (cTECs) and/or medullary thymic epithelial cells (mTECs). mTECs are characterized by cytokeratin 5 (K5) and cytokeratin 14 (K14) expression but low level of cytokeratin 8 (K8) expression, whereas cTECs express K8 and K18. In some embodiments, thymic cells are derived from TECs that express both K5 and K8 (K5+K8+). In some aspects, K5+K8+ cells are progenitors for mTECs and/or cTECs. mTECs may also be positive for the expression of Ulex europaeus agglutinin-1 (UEA-1) on cell surface, but not Ly51 (e.g., UEA-1+Ly51−), while cTECs are UEA-1−Ly51+. In some embodiments, thymic cells are or may be derived from mTECs.

In some embodiments, the population of thymic cells may include medullary thymic epithelial cells (mTECs). In some embodiments, the mTECs are derived by the differentiation of iPSCs. In some embodiments, mTECs may have high expression of markers such as, but not limited to, cytokeratin 5, cytokeratin 14, UEA-1, CD80, Cathepsin L, and/or Cathepsin S.

In some embodiments, the population of thymic cells may include cortical thymic epithelial cells (cTECs). In some embodiments, the cTECs are derived by the differentiation of iPSCs. In some embodiments, thymic cells are or may be derived from cTECs that have high expression of markers such as, but not limited to, cytokeratin 8, cytokeratin 18, Ly51, CD205, Cathepsin L and/or thymus-specific serine proteases. As a non-limiting example, thymic cells are or may be derived from cTECs that express markers such as, CCL25, and/or KRT5. mTECs may express markers such as, CCL19, KRT8, and/or AIRE.

In some embodiments, thymic cells are or may be derived from TECs that express one or more markers such as, FOXN1, PAX9, PAX1, DLIA, ISL1, EYA1, SIX1, IL7, K5, K8 and AIRE.

Thymic cells are or may be derived from any of the cell types described by Park et al. 2020 Science Vol. 367, Issue 6480 (the contents of which are herein incorporated by reference in its entirety). For example, thymic cells are derived from myoid cells, e.g., MYOD1 and MYOG expressing myoid cells (herein referred to as TEC(myo)) and/or from NEUROD1, SYP, CHGA-expressing TECS (herein referred to as TEC(neuro)).

Thymic cells are derived from the cell types described by Bautista et al. 2021 Nat Commun 12, 1096; the contents of which are herein incorporated by reference in its entirety. In some embodiments, the population of thymic cells derived by the differentiation of iPSCs may include “cTEC^{lo}” cells described by Bautista et al. 2021 and are characterized by lower levels of functional genes (HLA class II) and containing more KI67⁺-proliferating cells. Thymic cells are derived from “mTEC^{lo}” cells described by Bautista et al. 2021 and characterized by the expression of CLDN4, lower levels of HLA class II, expression of PSMB11, PRSS16, CCL25, and high levels of chemokine CCL21.

In some embodiments, the population of thymic cells derived by the differentiation of iPSCs may include “mTEC^{hi}” cells described by Bautista et al. 2021 and characterized by SPIB, AIRE, FEZF2, higher levels of expression of HLA class II. Thymic cells are or may be derived from corneocyte-like mTECs described by Bautista et al. 2021 and characterized by the expression of KRT1, and/or IVL

In some embodiments, the population of thymic cells derived by the differentiation of iPSCs may include immature TEC (iTEC) described by Bautista et al. 2021, which express canonical TEC identity genes e.g. FOXN1, PAX9, SIX1.

In some embodiments, the population of thymic cells derived by the differentiation of iPSCs may include TECs that express one or more markers, such as, but not limited to KRT5, KRT8, AIRE, PSMB11, and/or PRSS16.

In some embodiments, the population of thymic cells derived by the differentiation of iPSCs may include TECs that express one or more markers, such as, but not limited to AIRE, CK5, CK8, CXCL 12, CCL25, DLL4, and/or HLA-DR.

In some embodiments, the population of thymic cells derived by the differentiation of iPSCs may include cTEChi (or cTEC high) cells which are characterized by the expression of cell surface markers such as, but not limited to CTSV, SLC46A2, HLA-DMA, CXCL12, THY1, ENO1, CALR, ALCAM, ATPIF1, and/or HSPA5.

In some embodiments, the population of thymic cells derived by the differentiation of iPSCs may include AIRE+mTEC high cells which are characterized by the expression of one or more markers such as, but not limited to LTF, HLA-DRA<CD74, HLA DRB1, HLA-DPA, HLA-DPB1, IL2RG, and/or FCER2.

In some embodiments, the population of thymic cells derived by the differentiation of iPSCs may include corneocyte like mTECs. The cells are called corneocyte-like because they express genes such as keratin cytoskeletal 1 (KRT1), KRT10, SPINKS that are also expressed in corneocytes (terminally differentiated keratinocytes) of the skin. Corneocyte-like cells also express transcripts that overlap with mTEC, such as AIRE. Hence they are referred to as Corneocyte-like mTEC. They are likely the precursors that give rise to Hassall's' corpuscles, a unique cell in the human thymus. It is also thought that these cells are derived from mTEC precursor cells (Noam Kadouri et al Nature Review Immunology 2020 v20:239; the contents of which are herein incorporated by reference in its entirety).

### Pluripotent Stem Cells (PSCs)

In some embodiments, the cells of the present disclosure are derived from pluripotent stem cells.

Pluripotent stem cells have the capacity to give rise to any of the three germ layers: endoderm, mesoderm, and ectoderm. Pluripotent stem cells may comprise, for example, stem cells, e.g., embryonic stem cells, nuclear transfer derived embryonic stem cells, induced pluripotent stem cells (iPSC). The pluripotent stem cells may have a stem cell phenotype including (i) the ability to self-renew and (ii) pluripotency. Pluripotency-associated genes may include, but are not limited to, Oct-3/4, Sox2, Nanog, GDF3, REXI, FGF4, ESGI, DPPA2, DPPA4, hTERT and SSEAI.

Cells described herein may be derived from embryonic stem cells. ES cells may include a cell that (a) self-renews (b) differentiates to produce all cell types in an organism and/or (c) is derived from a developing organism. ES cells may be derived from the inner cell mass of the blastula of a developing organism. ES cells may also be derived from the blastomere generated by single blastomere biopsy (SBB) involving the removal of a single blastomere from the eight-cell stage of a developing organism. ES cells are characterized by the expression of markers such as, but not limited to SSEA-3, SSEA-4, TRA-1-60, TRA-1-81, and/or Alkaline phosphatase. Methods of generating and characterizing ES cells are known in the art and are found in, for example, U.S. Pat. Nos. 7,029,913; 5,843,780; 6,200,806 (the contents of each of which are herein incorporated by reference in their entirety).

Induced pluripotent stem cells (iPSCs) may also be used to generate cells of the present disclosure. iPSCs may include cells with one or more properties such as, but not limited to (a) self-renewal (b) ability to differentiate to produce all types of cells in an organism and/or (c) be derived from a somatic cell. iPSCs may express markers such as, but not limited to SSEA3, SSEA4, SOX2, OCT3/4, Nanog, TRA160, TRA1818, TDGF1, Dnmt3b, FoxD3, GDF3, Cyp26a1, TERT, Zpf42. Methods of generating and characterizing iPS cells are found in, for example, US Patent Publication Nos. US20090047263, US20090068742, US2009191159, US20090227032, US20090246875, and US20090304646 (the contents of each of which are herein incorporated by reference in their entirety). In some embodiments, the iPSCs are derived from a T cell or non-T cell, a B cell, or any other cell from peripheral blood mononuclear cell, a hematopoietic progenitor cell, or any other somatic cell type.

In some embodiments, pluripotent stem cells are derived from adult stem cells. Adult stem cells are obtained from the inner ear, bone marrow, mesenchyme, skin, fat, liver, muscle, and/or blood of a subject such as subject. PSCs may also include embryonic stem cells derived from a placenta or umbilical cord; progenitor cells (e.g., progenitor cells derived from the inner ear, bone marrow, mesenchyme, skin, fat, liver, muscle, and/or blood).

### Supporting Cells

In some embodiments, cells of the present disclosure may include or are cultured with supporting cells that aid in the generation of thymic cells and/or maintenance of thymic cells in culture. Non-limiting examples of supporting cells include hematopoietic non-T-cell progenitors, such as macrophages and dendritic cells (DCs); non-hematopoietic cells, such as epithelial cells and fibroblasts; stromal cells such as the progenitors of skeletal tissue, components such as bone, cartilage, the hematopoiesis-supporting stroma, and adipocytes. Supporting cells, in some embodiments, encourage the proliferation, survival, maturation, or function of thymic cells. In some embodiments, the supporting cells are mesenchymal in origin. In one embodiment, the mesenchymal cell are a mesenchymal stem cell.

Supporting cells are non-immune cells, that may be present in the thymic microenvironment. For example, the support cells are fibroblasts, vascular smooth muscle cells (VSMCs), endothelial cells, and/or lymphatic endothelial cells.

In some embodiments, the supporting cells are neuroendocrine cells (expressing BEX1, NEUROD1), muscle-like myoid (expressing MYOD1, DES), and myelin positive epithelial cells (expressing SOX10, MPZ) described by Bautista et al. 2021 Nat Commun 12, 1096 (2021); the contents of which are herein incorporated by reference in its entirety. In some embodiments, mesenchymal cells are associated with markers such as, but not limited to LAMA2, LAMA4, PDGFRA, PDGFRB, LUM, CSPG4, COL1A2, COL3A1, IGF1, FGF7, FGF10, FST, BMP4, SFRP2, WNT5A. The mesenchymal cells are positive or negative for one or more of these markers. In some embodiments, the mesenchymal cells are positive for some marks described herein but are negative for other markers.

In some embodiments, endothelial cells are associated with one or markers such as, but not limited to, VEGFC, PECAM1, APLNR, PROX1, LYVE1, ACKR1, SELE, SELP, FN1, and/or TGFB1. The endothelial cells are positive or negative for one or more of these markers. In some embodiments, the endothelial cells are positive for some marks described herein but are negative for other markers.

### Thymic Cell Products

In some embodiments, the present disclosure provides thymic cell products. The thymic cell products are prepared by the methods described herein. As used herein, a “thymic cell product” refers to a population of thymic cells that phenotypically or functionally bears resemblance to the human thymus or a sub population thereof, and/or suitable for a therapeutic, diagnostic, or a research application. The thymic products of the disclosure are prepared by (i) generating one or more population of thymic cells; (ii) extracting and sequencing RNA from each of the population of thymic cells; (iii) analyzing the RNA sequence to identify the sub populations within each population of thymic cells to generate a test thymic map for each of the population of thymic cells; (iv) comparing each of the test thymic maps to a reference thymic map; and (v) identifying a thymic cell product by selecting a test thymic map that has a Sub-Population Ratio (SPR) or a Relative cTEC Ratio (RCTR) of from about 0.7 to about 1.4. As used herein, SPR refers to the ratio of the proportion of a sub population (also herein a “cell type”) in a test thymic map to the proportion of a sub population of cells in a reference thymic map. As used herein, the cTEC ratio refers to the ratio of the proportion of cTEC high to cTEC low cells within a particular thymic map.

Thymic cell products are prepared by analyzing thymic maps. As used herein, a “thymic map” refers to the molecular profile of a population of thymic cells or thymic tissue. The molecular profile may include the total transcriptional profile of a population of thymic cells or thymic tissue; the sub populations of cells within the thymic cells or thymic tissue; and/or the transcriptional profile of the identified sub populations. In some embodiments, the thymic map are a reference thymic map, which, as used herein, refers to a representative map of the human thymus generated by transcriptional profile of thymus tissue and known cell types within the thymus tissue. In one embodiment, the reference thymic map may be generated using transcriptional profiling data described in Bautista et al. 2021. As used herein a test thymic map refers to characteristics of a population of thymic cells whose molecular profile is being compared to a reference thymic map.

In one embodiment, the SPR may be 0.70, 0.75, 0.8, 0.85, 0.9, 0.91, 0.92, 0.93, 0.94, 0.95, 0.06, 0.97, 0.98, 0.99, 1.0, 1.1, 1.2, 1.3, and/or 1.4. As a non-limiting example, the SPR is 1.

RCTR is prepared by calculating cTEC ratio. As used herein, the cTEC ratio refers to the ratio of the proportion of cTEC high to cTEC low cells within a particular thymic map (reference or test thymic map). While the cTEC ratio may vary from between thymic maps, RCTR may be 0.70, 0.75, 0.8, 0.85, 0.9, 0.91, 0.92, 0.93, 0.94, 0.95, 0.06, 0.97, 0.98, 0.99, 1.0, 1.1, 1.2, 1.3, and/or 1.4. In some embodiments, the RCTR may be from about 0.7 to about 1.14. As a non-limiting example, the RCTR may be 1. In some embodiments, the sub-populations of cells within each of the thymic cell populations may be immature thymic epithelial cells (iTECs), cTEC-high cells, cTEC-low cells, Aire+mTEC-high cells, mTEC-low cells, Corneocyte like mTEC cells, Ciliated cells, Myelin cells, Myoid cells, Neuroendocrine cells and/or Tuft/lonocyte cells. In some embodiments, the SPR may be from about 0.7 to about 1.4. In some embodiments, the SPR may be a iTEC SPR, a cTEC-high SPR, a cTEC-low SPR, a Aire+mTEC-high SPR, a mTEC-low SPR, a Corneocyte like mTEC SPR, a Ciliated SPR, a Myelin SPR, a Myoid SPR, a Neuroendocrine SPR and/or a Tuft/lonocyte SPR. In some embodiments, the SPR may be an iTEC SPR. As a non-limiting example, the iTEC SPR may be 1.

### III. Methods

The disclosure provides thymic cells, methods of making thymic cells and/or maintaining thymic cells in culture.

The present disclosure provides methods for generating thymic cells in vitro. These methods may include culturing a population of cells in the presence of a soluble factor, a mineral or a combination thereof. Such methods may induce differentiation or maturation of the population of cells to thymic cells. The population of cells may also be engineered to express a cell surface receptor or an intracellular factor. In some embodiments, the population of cells may be pluripotent stem cells (PSCs), definitive endodermal (DE) cells, third pharyngeal pouch endodermal (PPE) cells, anterior foregut endodermal (AFE) cells, thymic epithelial progenitor cell (TEPCs), immature thymic epithelial cells (iTECs), thymic epithelial cells (TECs), medullary thymic epithelial cells (mTECs), cortical thymic epithelial cells (cTECs) or combinations thereof.

In some embodiments, the thymic cell may be an immature thymic epithelial cell (iTEC). In some aspects, iTEC may express at least one cell surface receptor, and wherein the at least one cell surface receptor may be IGKC (ENSG00000211592), ANAX2, LIMA1 (ENSG00000050405), or EGFR (ENSG00000146648). In some embodiments, iTEC may expresses at least one intracellular factor, and wherein the at least one intracellular factor may be ASCL1 (ENSG00000139352), HES1 (ENSG00000114315), JUND (ENSG00000130522), FOS (ENSG00000170345), ARID5B (ENSG00000150347), IRF1 (ENSG00000125347), MAFB (ENSG00000204103), IF116 (ENSG00000163565), FOXC1 (ENSG00000054598), STAT1 (ENSG00000115415), JUNB (ENSG00000171223), EGFR1, ZFP36 (ENSG00000128016), JUN (ENSG00000177606), FOSB (ENSG00000125740), IER2 (ENSG00000160888), PAX9 (ENSG00000198807), or HIF1A (ENSG00000100644).

Soluble factors include, but are not limited to, growth factors, cytokines, transporters, hormones or combinations thereof.

As a non-limiting example the soluble factor may be a growth factor such as epidermal growth factor (EGF; ENSG00000138798).

The soluble factor may be a cytokine e.g. macrophage inhibitory factor (MIF; ENSG00000240972).

The soluble factor may also be a transporter e.g., Lactotransferrin (LTF; ENSG00000012223).

In some embodiments, the cells of the disclosure may be cultured in the presence of a mineral. The mineral may be iron, magnesium, calcium, manganese, molybdenum, phosphorus, potassium, sodium, sulfur, zinc, chloride, chromium, copper, fluoride or iodine. In one aspect, the mineral may be iron.

The cells of the disclosure may be engineered to express a cell surface receptor or an intracellular factor for the purpose of generating or maintaining thymic cells. The cell surface receptor may be CD74 (ENSG00000019582), integrin beta 1 (ITGB1; ENSG00000150093) and/or epidermal growth factor receptor (EGFR; ENSG00000146648).

The intracellular factor may be ENO1 (ENSG00000074800).

The present disclosure also provides methods of maintaining a population of thymic cells in vitro. The methods may include (i) culturing the population of thymic cells in the presence of a soluble factor or a mineral; (ii) culturing the population of thymic cells in the presence of one or more supporting cells; and/or (iii) engineering the population of thymic cells to express a cell surface receptor or an intracellular factor.

The cells of the disclosure may be cultured in the presence of one or more supporting cells. Supporting cells may be endothelial cells, mesenchymal cells, macrophages, dendritic cells (DCs), epithelial cells, fibroblasts, stromal cells, adipocytes, fibroblasts, vascular smooth muscle cells (VSMCs), or lymphatic endothelial cells. In some embodiments, supporting cells may be mesenchymal cells. In some aspects, the supporting cells may be endothelial cells.

### Preparation and Maintenance of Thymic Cells

In some embodiments, the present disclosure provides methods for the preparation of one or more cells or cell types described herein. In some embodiments, the cells may be thymic cells.

An accumulating body of data in public databases provide single cell transcriptomes of primary human and murine thymuses (See Bautista et al. 2021 Nat Commun 12, 1096; Kernfeld, et al. Immunity. 2018 Jun. 19; 48(6):1258-1270.e6; Zeng et al. Immunity. 2019 Nov. 19; 51(5):930-948.e6; the contents of each of which are herein incorporated by reference in its entirety). This provides a rich source of material for identification of factors that promote the differentiation and/or maturation of cells of the disclosure to thymic cells. By analysis of scRNA sequencing data, the present disclosure identifies potential factors and/or supporting cells that may promote and/or maintain thymic cell phenotype.

In some embodiments, cells of the present disclosure may be isolated from an organism. In some embodiments, the organism may be a mammal. Mammalian cells may be isolated from human, rodent, porcine and/or bovine sources. Human sources of cells of the disclosure may be autologous or allogeneic. In some embodiments, the tissues that contain the cells of the disclosure may be harvested and used as such for applications described herein. Cells of the disclosure may be obtained from embryonic, fetal, adult organism. In some aspects, the organism may be alive or may be a cadaver organism.

Cells described herein may be derived from other cell types. As a non-limiting example, the cells of the disclosure may be derived from pluripotent stem cells (PSCs). In some embodiments, the cells of the disclosure may be derived from progenitor cells. In some embodiments, cells of the disclosure may be derived by the differentiation of PSCs and/or progenitor cells.

In some embodiments, thymic cells may be prepared from PSCs. In this regard, the methods may comprise culturing the pluripotent stem cells for a time and under conditions sufficient to differentiate the pluripotent stem cells into thymic cells. For example, the method may comprise culturing the pluripotent stem cells in the presence of the factors and/or inhibitors that drive the differentiation of PSCs to thymic cells. Methods for differentiating PSCs into thymic cells are known in the art. The methods for differentiating PSCs into thymic cells may include the use of one or more parameters know in the art for differentiation or combinations thereof. The parameters include, but are not limited to, (i) factors promoting differentiation (ii) inhibitors promoting differentiation (iii) duration of time for promoting differentiation (iv) temperature (v) substrate and/or (vi) supporting cells that promote differentiation. Any of the methods or parameters for differentiating PSCs into thymic cells described in following references may be used herein and include Parent et al. Cell Stem Cell. 2013 Aug. 1; 13(2):219-29; Soh et al. Stem Cell Rep. 2014 Vol. 2 j 925-937; Sun et al. Cell Stem Cell. 2013 Aug. 1; 13(2):230-6; Okabe et al. Cell. Reprog. 2015 Vol 17, No. 5; Su et al. Sci. Rep. 2015 5, 9882; Otsuka et al. Sci Rep 2020 10:224; International Patent Publications, WO2019060336, WO2020205859, WO2020220040, WO2014134213, WO2010143529, WO2011139628; and Chinese Patent Publication CN201110121243; the contents of each of which are herein incorporated by reference in their entirety.

### Soluble Factors

Factors described herein may be used to guide maturation, fate specification, and/or aid in the maintenance of certain cells states of the cells of the disclosure. Soluble factors may include a protein or peptide that can bind to a cell surface molecule or be taken up by a cell. Uptake of the soluble factor by the cell may be by passive diffusion, by a transporter, and/or by endocytosis. The soluble factors may include growth factors, hormones, transporter, and cytokines. When provided to a cell, the soluble factor may generate a signal to the cell. The signal may result in one or more cell actions such as survival, proliferation and differentiation.

In some embodiments, the soluble factor may be a ligand.

In some embodiments, the soluble factor may be growth factor. The growth factor may be epidermal growth factor (EGF), fibroblast growth factor (FGF), nerve growth factor (NGF), platelet derived growth factor (PDGF), vascular endothelial growth factor (VEGF), insulin-like growth factor (IGF), granulocyte-macrophage colony-stimulating factor (GMCSF), granulocyte colony-stimulating factor (GCSF), transforming growth factors (TGFs), multiplication-stimulating factor (MSF), Erythropoietin, Thrombopoietin (TPO), bone morphogenetic protein (BMP), hepatocyte growth factor (HGF), GDF, Neurotrophin, sarcoma growth factor (SGF), and/or growth/differentiation factors (GDFs).

As a non-limiting example, the growth factor may be epidermal growth factor (EGF; ENSG00000138798).

In some embodiments, the soluble factor may be a cytokine. Any of the cytokines known in the art may be utilized. As a non-limiting example, the cytokine may be produced by a thymic cell or an effector cell in vivo. Yan F, et al. Mol Med Rep 16: 7175-7184, 2017 provide cytokines generated by T cells or thymic cells which may be useful in the present disclosure (the contents of which are herein incorporated by reference in its entirety). As a non-limiting example, the cytokine may be macrophage inhibitory factor (MIF; ENSG00000240972).

In some embodiments, the soluble factor may be a transporter protein, which may bind to a second companion protein or mineral and facilitate its transfer from one location to another location. The transfer may occur from an extracellular environment to within a cell, or from one location within the body of the organism to another. In some embodiments, the transporter protein may be a mineral transporter protein. As a non-limiting example, the transporter protein may be Lactotransferrin (LTF; ENSG00000012223).

### Minerals

In some embodiments, cells of the disclosure may be prepared or cultured in the presence of a mineral. The mineral may be iron, magnesium, calcium, manganese, molybdenum, phosphorus, potassium, sodium, sulfur, zinc, chloride, chromium, copper, fluoride or iodine. In some embodiments, the mineral may be iron, or a salt or derivative thereof.

### Cell Surface Receptors

Soluble factors or ligands may interact with a counterpart receptor that is specific to the ligand and together transmit a message or signal to the cells to take on a particular function or phenotype. In some embodiments, the cell may be a thymic cell. Cell surface receptor may be a G protein-coupled receptors, enzyme-coupled receptors, and ion-channel-linked receptors.

Non-limiting examples of cell surface receptors include, CD74 (ENSG00000019582), integrin beta 1 (ITGB1; ENSG00000150093) and/or epidermal growth factor receptor (EGFR; ENSG00000146648). In one aspect, the cell surface receptor may be CD74. The CD74 has been demonstrated to regulate cellularity and maturation of thymic cells by activating NF kappa B (Wang et al. FASEB J. 2021 May; 35(5):e21535; the contents of which are herein incorporated by reference in its entirety).

### Intracellular Factors

Methods of the present disclosure may include culturing populations of cells described herein with one or more intracellular factors. The intracellular factor may be any protein or peptide that is located in the cytoplasm or the nucleus.

In some embodiments, the intracellular factor is a cytoplasmic intracellular factor. As a non-limiting example, the intracellular factor may be intracellular factor is Enolase 1 (ENO1; ENSG00000074800). In other aspects, the intracellular actor may be a factor residing in the nucleus. For example, the factor may be a transcription factor or a transcription associated factor. Non-limiting examples of intracellular nuclear factors may include NFKB1, PAX1, PRDX15, PSIP1, AIRE, DLX5, FEZF2, LTF, SPIB.

### Analysis of Thymic Cell Populations

Thymic cell populations derived by the differentiation of stem cells may be analyzed by one or more methods described herein. In some embodiments, thymic cell populations may include one or more of the cell types such as, but not limited to cTEC-hi, cTEC-lo, Immature TEC, mTEC-lo, Aire+mTEC hi, Keratinocyte-like mTEC, Neuroendocrine cells, Myoid cells, Myelin cells. Each of the thymic cell type may be associated with one or more markers. In some embodiments the cTEC-hi cells may be associated with the expression of markers, such as, but not limited to, PSMB11, PRSS16, and CCL25. In some embodiments the cTEC-lo cells may be associated with the expression of markers, such as, but not limited to, PSMB11, PRSS16, CCL25 (with lower levels of HLA class II, PSMB11, PRSS16, CCL25; increased K167+ proliferating cells). In some embodiments the immature TEC cells may be associated with the expression of markers, such as, but not limited to, FOXN1, PAX9, SIX1, lacking cTEC/mTEC functional genes. In some embodiments the mTEC-lo cells may be associated with the expression of markers, such as, but not limited to, CLDN4, lower levels of HLA class II, high levels of the chemokine CCL21. In some embodiments the Aire+mTEC hi cells may be associated with the expression of markers, such as, but not limited to, SPIB, AIRE, FEZF2, higher levels of HLA class II. In some embodiments the Keratinocyte-like mTEC cells may be associated with the expression of markers, such as, but not limited to, KRT1, IVL. In some embodiments the Neuroendocrine cells may be associated with the expression of markers, such as, but not limited to, BEX1, NEUROD1. In some embodiments the Myoid cells may be associated with the expression of markers, such as, but not limited to, MYOD1, DES. In some embodiments the Myelin cells may be associated with the expression of MPZ. As used throughout this description, reference to “low” or “lower” levels of marker gene expression in a subpopulation of cells means about 1.5-fold lower, about 2-fold lower, about 2.5-fold lower, about 3-fold lower, about 3.5-fold lower, about 4-fold lower, about 4.5-fold lower, or about 5-fold lower, or lower than 5-fold lower than mean expression levels of the marker gene across a population of cells. Similarly, throughout this description, reference to “high” or “higher” levels of marker gene expression in a subpopulation of cells means about 1.5-fold higher, about 2-fold higher, about 2.5-fold higher, about 3-fold higher, about 3.5-fold higher, about 4-fold higher, about 4.5-fold higher, or about 5-fold higher, or higher than 5-fold higher than mean expression levels of the marker gene across a population of cells.

In some embodiments, CellRouter may be used to perform quality control, cell cycle, and mitochondrial content regression (to account for potential confounding effects of cell cycle and stressed cells), followed by identification of variable genes, dimensionality reduction (UMAP=Uniform Manifold Approximation and Projection) and clustering as described in da Rocha et al. Nature Communications. 2018 Mar. 1; 9(1):892 (the contents of which are herein incorporated by reference in their entirety). CellRouter may be used to identify transcriptional clusters in thymic cell populations. In some embodiments, single-cells and clusters may be visualized in the UMAP space. Canonical marker genes of several lineages may be used to assign a cell type identity to each transcriptional cluster.

In some embodiments, scRNA sequence data of thymic cells derived in vitro or obtained in vivo from an organism may be compared. Large-scale single-cell transcriptomic datasets generated using different technologies contain batch-specific systematic variations that present a challenge to data integration. Methods of batch correction may include regression-based batch correction (da Rocha et al. Nature Communications. 2018 Mar. 1; 9(1):892 (the contents of which are herein incorporated by reference in their entirety) and harmony-based batch correction (Korsunsky et al. Nature Methods; 16, pages 1289-1296 (2019).

In some embodiments, the batch correction may be performed using Single Cell Net, Symphony (Kang, J. B., et al. Nat Commun 12, 5890 (2021); the contents of which are herein incorporated by reference in its entirety) and Label Transfer (as implemented in the package Seurat (Stuart et al., 2019, Cell 177, 1888-1902; the contents of which are herein incorporated by reference in its entirety).

### IV. Definitions

Expression: As used herein, “expression” and grammatical equivalents thereof, in the context of a marker, refers to production of the marker as well as level or amount of the marker. For example, expression of a marker or presence of a marker in a cell or a cell is positive for a marker, refers to expression of the marker at a level that is similar to a positive control level. The positive control level may be determined by the level of the marker expressed by a cell known to have the cell fate associated with the marker. Similarly, absence of expression of a marker or a cell is negative for a marker, refers to expression of the marker at a level that is similar to a negative control level. The negative control level may be determined by the level of the marker expressed by a cell known to not have the cell fate associated with the marker. As such, absence of a marker does not simply imply an undetectable level of expression of the marker, in certain cases, a cell may express the marker but the expression may be low compared to a positive control or may be at a level similar to that of a negative control.

Effector cell: As used herein, an “effector cell” refers to any cell or cell type which, when in contact with or in proximity to a thymic cell, acquires the ability to execute, initiate or propagate a signal or a cell death trigger. “Contact or proximity” refers to spatiotemporal closeness sufficient to enable cell-intrinsic or cell-extrinsic (e.g., cell-to-cell) signaling or other communication or interaction.

Lymphocyte: As used herein, a “lymphocyte” embraces the meanings and uses that a person of ordinary skill in the art would understand the term to embrace, and additionally refers to a type of immune cell originating in the bone marrow that resides in lymphoid tissues or blood. In some embodiments, lymphocytes undergo maturation in the thymus.

Negative: As used herein, the term “negative” (which may be abbreviated as“−”), as used herein with reference to expression of the indicated cell marker, means that the cell does not express the indicated cell marker at a detectable level.

Positive: As used herein, the term “positive” (which may be abbreviated as “+”) with reference to expression of the indicated cell marker, means that the cell expresses the indicated cell marker at any detectable level, which may include, for example, expression at a low (but detectable) level as well as expression at a high (hi) level.

Pre-T cell: As used herein, a “pre-T cell” refers to a lymphocyte that is capable of maturing or differentiating into a T cell.

Soluble factor: As used herein, a “soluble factor” refers to any protein or peptide that can bind to a cell surface molecule or be taken up by a cell. Uptake by the cell may be by passive diffusion, by a transporter, and/or by endocytosis.

Sub-Population Ratio (SPR): As used herein, SPR refers to the ratio of the proportion of a sub population of thymic cells (also herein a “cell type”) in a test thymic map to the proportion of a sub population of thymic cells in a reference thymic map. In some embodiments, the SPR may be a iTEC SPR, a cTEC-high SPR, a cTEC-low SPR, a Aire+mTEC-high SPR, a mTEC-low SPR, a Corneocyte like mTEC SPR, a Ciliated SPR, a Myelin SPR, a Myoid SPR, a Neuroendocrine SPR or a Tuft/lonocyte SPR.

cTEC Ratio and Relative cTEC Ratio (RCTR): As used herein, the cTEC ratio refers to the ratio of the proportion of cTEC high to cTEC low cells within a particular thymic map (reference or test thymic map). RCTR refers to the ratio of cTEC Ratio in a test thymic map to a reference thymic map.

Thymic cell or origin or lineage: As used herein, “thymic cell or thymic origin or thymic lineage” refers to a cell with one or more phenotypic or genotypic markers associated with a cell derived from the thymus or a cell destined to become a cell of the thymus. As used herein, the thymus may be an embryonic, a fetal or an adult thymus.

Thymic cell product: As used herein, a “thymic cell product” refers to a population of thymic cells that phenotypically or functionally bears resemblance to the human thymus or a sub population thereof, and/or suitable for a therapeutic, diagnostic, or a research application.

Thymic map: As used herein, a “thymic map” refers to the molecular profile of a population of thymic cells or thymic tissue. The molecular profile may include the total transcriptional profile of a population of thymic cells or thymic tissue; the sub populations of cells within the thymic cells or thymic tissue; and/or the transcriptional profile of the identified sub populations. In some embodiments, the thymic map may be a reference thymic map, which, as used herein, refers to a representative map of the human thymus generated by transcriptional profile of thymus tissue and known cell types within the thymus tissue. In one embodiment, the reference thymic map may be generated using transcriptional profiling data described in Bautista et al. 2021. As used herein a test thymic map refers to characteristics of a population of thymic cells whose molecular profile is being compared to a reference thymic map.

Variant: The term “variant” as used in reference to a biomolecule (e.g., a training factor or a terminal factor) refers to a biomolecule that is related to or derived from a parent molecule. The variant can be, for example, a modified form, a truncated form, a mutated form, a homologous form, or other altered form of the parent molecule. The term variant can be used to describe either polynucleotides or polypeptides.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLES

### Example 1. Single Cell RNA Sequencing Analysis of Thymic Cells

Analysis of the scRNA-seq data of thymuses of 19-day fetus and 25-year-old adult as published in Bautista, J. et al. was performed (Single-cell transcriptional profiling of human thymic stroma uncovers novel cellular heterogeneity in the thymic medulla. Nat Commun 12, 1096 (2021); the contents of which are herein incorporated by reference in its entirety).

CellRouter was used to perform quality control, cell cycle, and mitochondrial content regression (to account for potential confounding effects of cell cycle and stressed cells), followed by identification of variable genes, dimensionality reduction (UMAP) and clustering as described in da Rocha et al. Nature Communications. 2018 Mar. 1; 9(1):892 (the contents of which are herein incorporated by reference in their entirety). CellRouter identified 24 transcriptional clusters. Single-cells and clusters were visualized in the UMAP space. Canonical marker genes of several lineages were used to assign a cell type identity to each transcriptional cluster. A heatmap which shows the mean expression of each gene in each cluster was generated

Five mesenchymal cell (MC) states were identified (designated as MC1, MC2, MC3, MC4 and MC5). Further unbiased cell-cell communication analysis identified mesenchyme-epithelium interactions, for example between MC2 and mTEC.

Cell-cell communications/interactions were visualized as a heatmap to analyze the degree of interaction between different cells. Unbiased cell-cell communication analysis identified mesenchyme-epithelium interactions, especially between MC2 and mTEC. Cells designated as Endo1 and Endo3 appeared to interact with certain TEC subsets. Identification of such interactions may facilitate the identification of interacting and regulatory gene candidates.

Endothelial cells were found to strongly interact with TEC cells, particularly, mTECs and have prioritized ligand-receptor interactions which lead to signalling pathways being reported as important for the development of thymic epithelial cells and the cell types potentially expressing the ligands. One such example is the EGF receptor (EGFR) pathway in cTECs and mTECs These data highlight the potential utility of using EGF in thymic cell culture. Cell types were expressing ligand were paired with cell type expressing receptors and analyzed. A strong interaction between endothelial cells and TEC cells (particularly mTECs) was identified. Receptors such as EGFR, ITGB1 were identified in the analysis.

Further prioritization of ligand-receptor interactions led to the identification of signalling molecules and networks that may be important for the development of thymic epithelial cells (e.g. MIF and CD74) and potential effectors (e.g. CD74 to LTF). Activity score were used to study the enrichment of cell type-specific gene signatures in gene sets predicted to be controlled by the reported transcriptional regulators based on gene regulatory network analysis.

Matching of receptors and downstream effectors identified putative signalling molecules and networks mediating the activity of receptors. Transcriptional regulators responsive to specific cell surface receptors that could be important for the development of TECs were also identified. For example, LTF and ENO-1 which are responsive to CD74 were identified. Eno-1 (Enolase) is known to be important for the metabolism of T cells. LTF (Lactoferrin) is involved in iron transport. These findings suggest that the utility of iron supplementation in TECs cultured in vitro. Since MIF (Macrophage Inhibitory Factor) is a known ligand for CD74, this finding shows the utility of the addition of MIF to the induce differentiation of one or more cell type to a thymic cell and/or in the maintenance of thymic cells.

### Example 2. Comparison of Fetal and Adult Thymi

Comparison of the transcriptional profile of young (or herein referred to as fetal) (19-week) versus old (25-year-old) thymuses (or thymi) showed strikingly different profiles. The transcriptional profile of the young thymus showed multiple sub populations including mTEC high, cTEC high, mTEC low, cTEC low, iTEC, neuroendocrine, endothelial cells (Endo1, Endo2, Endo3, Endo4, Endo5), mesenchymal cells (MC1, MC2, MC3, MC4, MC5), and pericytes (Pericyte-1, Pericyte-2, Pericyte-3). In contrast, the transcriptional profile of the old thymus showed sub populations of endothelium, epithelium, pericyte and mesenchymal cells. The abundance of epithelial cells was substantially reduced in the adult thymus (25 years old) compared to the fetal thymus (19 weeks old). The epithelial cells also appear to have immature TECs. Endothelial cell subsets, as well as some pericyte subsets, appeared to be conserved between 19 weeks and 25 years old thymus, while mesenchymal cell subsets appeared distinct between ages. These data show that the deficiency of thymus occurs even at the age of 25 years.

### Example 3. Transcriptional Regulators

Using an algorithm for following trajectory of cell differentiation, showed different kinetics of development of mTEC versus cTEC from early iTEC. Each of the trajectories highlighted certain prominent gene networks, signaling molecules and regulators that could contribute to the differentiation to either mTEC or cTEC. Kinetic patterns of prioritized transcriptional regulators showed different pattern of differentiation from iTEC to cTEC compared to iTEC to mTEC.

### Example 4. Comparison of scRNA Sequence Datasets of Thymic Tissue and iPSC Derived Thymic Cells

scRNA sequence data of thymic tissue from Bautista, J. et al. Nat Commun 12, 1096 (2021) was compared to scRNA sequence data of thymic tissue from Park et al. 2020 Science Vol. 367, Issue 6480 (the contents of which are herein incorporated by reference in its entirety). Large-scale single-cell transcriptomic datasets generated using different technologies contain batch-specific systematic variations that present a challenge to data integration. Without accounting for the differences that arise from batch variation, the data clustered according to the age of the thymic tissue (fetal vs. adult) rather than by cell type. Two methods of batch correction were performed namely regression-based batch correction (da Rocha et al. Nature Communications. 2018 Mar. 1; 9(1):892 (the contents of which are herein incorporated by reference in their entirety) and harmony-based batch correction (Korsunsky et al. Nature Methods; 16, pages 1289-1296 (2019). These data showed that the harmony-based batch correction was more effective in clustering the data between the two studies compared to regression-based batch correction.

To quantify cell similarities in the different populations in fetal and adult thymi identified in the scRNA seq data in Bautista et al. and Park et al., a Mean Classification Score (MCS) was employed.

Using cell types from Park et al (herein referred to as the “Teichmann study”) machine-learning models were trained to identity cell types using single-cell gene expression profiles and the SingleCellNet algorithm (Tan Y, et al. Cell Syst. 2019 Aug. 28; 9(2):207-213.e2; the contents of which are herein incorporated by reference in its entirety). These models therefore represent the identity of the cell types given their transcriptome. These models were then queried to identify what, if any, transcriptome similarity existed across cell types in the dataset included in Bautista et al. (herein referred to as the “Parent” dataset). To facilitate visualization, mean classification of scores were calculated. Each single cell sub population from Parent dataset has a score and the heatmap shows the mean score in previously annotated cell type from Parent's data. Classification scores range from 0 to 1, with 0 indicating no similarity and 1 indicating high similarity. Subpopulations described in Teichmann data set were identified with high MCS in Parent dataset. The observed conservation of cell types between both studies identified after batch correction represents an important step in harmonizing published scRNA sequence data sets of thymic tissue. Insights obtained from analysis of these data sets are then used to characterize thymic populations derived from the differentiation of pluripotent stem cells.

scRNA sequencing was performed on thymic cells differentiated from pluripotent stem cells. 7 subpopulations of cells were identified from these TEP derivations). The scRNA sequence data from the in vitro derived thymic cells derivations was compared to the scRNA sequence data set in Bautista et al. and mean classification scores were calculated. This comparison provides an insight into the cell populations present within the in vitro derivations and their correlation with cell types present in vivo in thymic tissue. The mean classification score based heat map revealed populations in the TEP derivations with strong similarity to neuro-endocrine cells (populations 5, 3, and 4) and to comeocyte-mTEC like cells (populations 1, and 2 and populations 6, 7) with less similarity to cTEC-low, ionocyte and Myelin cells. The enrichment of neuroendocrine cells in the population could likely be due to the presence of only 45% definitive endoderm cells and the differentiation to neuroendocrine cells may be promoted by SMAD inhibition during definitive endoderm to anterior foregut differentiation. The neuroendocrine cells may also be neuronal cells that develop a neuroendocrine phenotype. The presence of corneocyte like mTECs in the derivations is desirable. TEP derivation conditions may be tuned and culture conditions that support the enrichment of iTECs as determined by comparison of scRNA seq data may be desired.

Corneocyte-like cells-similar to mTEC (population 1, 2): This population was identified in Bautista et al 2021 and based on the analysis herein may be equivalent to mTEC III described by Park et al. 2020. The cells are called corneocytes because they express genes such as Keratin cytoskeletal 1 (KRT1), KRT10, SPINKS that are also expressed in corneocytes (terminally differentiated keratinocytes) of the skin. Corneocyte-like cells they also express transcripts that overlap with mTEC, such as AIRE. Hence they are referred to as Corneocyte-like mTEC. They are likely the precursors that give rise to Hassall's' corpuscles, a unique cell in the human thymus. It is also thought that these cells are derived from mTEC precursor cells (Noam Kadouri et al Nature Review Immunology 2020 v20:239; the contents of which are herein incorporated by reference in its entirety). Populations 1 and 2 that resemble corneocyte also appear to have some similarities to Tuft/Ionocyte cells (Miller C et al 2018 Nature 559:627; the contents of which are herein incorporated in its entirety). Thymic tuft cells were shown to localize adjacent to corneocyte-like (Kadouri N, et al. Nat Rev Immunol. 2020 April; 20(4):239-253; the contents of which are herein incorporated in its entirety).

### Example 5. Thymic Cell Surface Receptor and Transcriptional Regulators

One of the objectives of the comparative analysis of scRNA sequence is to identify progenitor or precursors of cells that ultimately give rise to TEP cells during differentiation of pluripotent stem cells in vitro. The presence of “bi-potent” progenitor cells also referred to as iTEC cells that give rise to both mTEC and cTEC population has been speculated in the field but not definitively identified. Computational tracing of cellular connectivity conducted herein suggest that the iTEC population may give rise to the “cTEC^{lo}” and “mTEC^{lo}” cells that develop further into “cTEC^{high}” and “mTEC^{high}” cells. To aid in recognizing subpopulations of thymic cells, surface markers and transcriptional factors preferentially expressed in the various subpopulations have been identified. Notably the analysis identified surface markers and transcriptional factors for two groups of iTEC cells. Cell surface markers include IGKC (ENSG00000211592) for iTEC-1 and ANAX2, LIMA1 (ENSG00000050405), EGFR (ENSG00000146648) for iTEC-2. Transcriptional regulators include ASCL1 (ENSG00000139352), HES1 (ENSG00000114315), JUND (ENSG00000130522), FOS (ENSG00000170345), ARID5B (ENSG00000150347), IRF1 (ENSG00000125347), MAFB (ENSG00000204103), IFI16 (ENSG00000163565), FOXC1 (ENSG00000054598), STAT1 (ENSG00000115415) for iTEC-1 and JUNB (ENSG00000171223), EGFR1, ZFP36 (ENSG00000128016), JUN (ENSG00000177606), FOSB (ENSG00000125740), IER2 (ENSG00000160888), PAX9 (ENSG00000198807), HIF1A (ENSG00000100644).

### Example 6. Workflow for Cell Identity Analysis

The objective of this study was to develop a workflow using algorithms to determine the molecular similarity of iPSC-derived thymic cells relative to reference cell atlases published in Bautista et al. 2021 and Park et al. 2020. Analysis of iPSC-derived thymic cells is often challenging due to substantial heterogeneity and asynchronous cell behaviors during directed differentiation of iPSCs towards target cell types. Employing orthogonal and complementary approaches to determine the molecular similarity of in vitro iPSC-derived thymic cells relative to their in vivo counterparts is critical since algorithms are built upon different assumptions and might better capture some aspects of the data while missing others. The workflow utilized SingleCellNet, Symphony (Kang, J. B., et al. Nat Commun 12, 5890 (2021); the contents of which are herein incorporated by reference in its entirety) and Label Transfer (as implemented in the package Seurat (Stuart et al., 2019, Cell 177, 1888-1902; the contents of which are herein incorporated by reference in its entirety). The algorithms were used to classify cells from iPSC-derived thymic cells (SingleCellNet, map iPSC-derived cells to a reference UMAP and classify cells based on class labels derived from the training dataset (Symphony) and transfer labels learned from the training dataset to individual cells in the query dataset.

The workflow was applied to thymic cell populations differentiated from iPSC cells (also referred herein as “iPSC derived thymic cells”). Two iPSC derived thymic cell population samples were used namely, 2-179 and Exp 21 iPS-TEP. First, the Symphony algorithm was applied to project query cells (sample 2-179) and to the reference UMAP cell atlas generated by Bautista et al. 2021 such that query and training cells are located in similar positions in the UMAP space (UMAP=Uniform Manifold Approximation and Projection). Cell identity was also determined based on a specific criteria using this approach. Consistent with the SingleCellNet analysis, Symphony also assigned a subset of query cells to the neuroendocrine state. However, the corneocyte-like similarity was not captured by Symphony, which determined the majority of iPSC-derived cells from sample 2-179 as cTEC-low or cTEC-high. The Label Transfer approach (available in the software package Seurat) was also utilized, in which class labels (cell types) learned from the training dataset e.g. Bautista et al. 2021 were transferred to query cells e.g. samples 2-179 and 4-191. Following a UMAP analysis of iPSC sample 2-179 (query), labels were transferred from the reference to the query cells. Symphony and Seurat analysis showed good agreement with regard to assignment of the neuroendocrine and cTEC-low or cTEC-high cell populations.

Next, sample Exp 21 iPS-TEP was analyzed using Symphony, also showing previous results from SingleCellNet, to establish comparisons. Symphony analysis demonstrated an increased number of neuroendocrine cells when compared to the same analysis of sample 2-179 (consistent with SingleCellNet results), but with a higher number of cTEC-low cells. According to these predictions, the numbers of cTEC-high cells is smaller, while Myelin and Myoid cells were substantially more abundant in this sample, which is in partial agreement with the SingleCellNet analysis. Additionally, using Symphony to analyze Exp21 iPS-TEP an i-TEC population was detected.

The percentage frequency of different cell types between the thymic cell derivations (Experiment 21)—the Query, were compared to the frequency of cell types identified in the primary human thymus by Bautista et al. 2021—the Reference. This comparison revealed that the percentage of neuroendocrine cells is higher in the Query compared to the Reference. Aire+mTECs were only present in the Reference but not the Query. Whereas, the iTEC and cTEC high population frequency in Query was quite similar to iTEC frequency in Reference. Thus, cell types identified in TEPs derived from iPS cells was comparable in frequency to the cell types identified in primary human thymus tissue. The number of cells for each cell type in Query and Reference is provided in Table 1.

TABLE 1
Cell numbers in Query and Reference

| Cell type | Query | Reference |
|---|---|---|
| AIRE + mTEC-high | 0 | 183 |
| Ciliated cells | 174 | 239 |
| Corneocyte-like | 11 | 941 |
| mTEC | | |
| cTEC-high | 1600 | 3827 |
| cTEC-low | 770 | 4460 |
| iTEC | 599 | 1447 |
| mTEC-low | 18 | 718 |
| Myelin | 10 | 426 |
| Myoid | 51 | 562 |
| Neuroendocrine | 1263 | 591 |
| Tuft/Ionocyte | 214 | 170 |

### Example 7. Recapitulating Thymic Diversity in iPSC Derived Thymic Cells

A reference single-cell atlas of epithelial cells from the human thymus (also herein “reference thymic map” described in Bautista et al. 2021 was generated using the algorithms CellRouter and Symphony. iPSC-derived thymic cells were mapped onto the reference single-cell atlas, and iPSC-derived thymic cells were classified based on their molecular similarity to their in vivo counterparts in the human thymus to generate a test thymic map. As used herein, “molecular similarity” may be defined as a classification score, which is calculated by a supervised machine learning algorithm termed a classifier, with scores close 0 meaning a low probability of a given cell to be similar to a particular cell type, while scores close to 1 indicate a high probability of a given cell to be similar to their in vivo counterparts in the human thymus.

This approach indicated that in vitro protocols to differentiate thymic epithelial cells from iPSCs (e.g. Experiment 21 and Experiment 23), were better at recapitulating the cell type diversity of the human thymus (data not shown). Investigating the proportion of cell types derived from iPSC cells across samples revealed that Experiments 21 and 23 contain cells transcriptionally similar to critical cell types in the thymus, such as cTECs (including cTEC-high, cTEC-low), a subset of mTECs, immature TECs, and other cell types (see Table 2 below).

TABLE 2
Proportion of different cell types derived from iPSC cells

| | | | | | | | | |
|---|---|---|---|---|---|---|---|---|
| Human | Exp | Exp | Exp | Exp 21 | Exp 21 | Exp 23 | Exp 23 | |
| thymus | 7A | 7B | 7C | Rep 1 | Rep 2 | Rep 1 | Rep 2 | SBR |

| | | | | | | | | |
|---|---|---|---|---|---|---|---|---|
| Aire+ mTEC-high | 1.3 | 0 | 0 | 0 | 0 | 0 | 0.9 | 2 |
| Ciliated | 1.8 | 1.8 | 2.5 | 1.3 | 2.6 | 3.7 | 0.2 | 0.2 |
| Corneocyte like mTEC | 6.9 | 0.0 | 0.0 | 0.0 | 0.1 | 0.1 | 0.8 | 0.5 |
| cTEC-high | 28.2 | 80.7 | 4.4 | 58.4 | 37.1 | 40.1 | 45.1 | 45.6 |
| cTEC-low | 32.9 | 4.9 | 51.1 | 13.9 | 15.2 | 10.0 | 40.1 | 40.6 |
| iTEC | 10.7 | 1.4 | 4.5 | 2.7 | 15.6 | 14.3 | 2.8 | 2.8 |
| mTEC-low | 5.3 | 0.0 | 0.0 | 0.0 | 0.1 | 0.1 | 0.0 | 0.1 |
| Myelin | 3.1 | 0.1 | 0.6 | 0.1 | 0.3 | 0.3 | 0.3 | 0.2 |
| Myoid | 4.1 | 0.1 | 4.4 | 0.4 | 0.7 | 0.7 | 0.3 | 0.2 |
| Neuroendocrine | 4.4 | 10.2 | 32.0 | 22.4 | 26.7 | 28.2 | 8.9 | 8.4 |
| Tuft/lonocyte | 1.3 | 0.9 | 0.6 | 0.8 | 1.8 | 2.4 | 0.7 | 0.6 |

The Sub-Population Ratio (SPR) was calculated for the samples in Table 2 and the results are presented in Table 3. As used herein, SPR refers to the ratio of the proportion of a sub population (also herein a “cell type”) in a test thymic map to the proportion of a sub population of cells in a reference thymic map. In some embodiments, the SPR may be a iTEC SPR, a cTEC-high SPR, a cTEC-low SPR, a Aire+mTEC-high SPR, a mTEC-low SPR, a Corneocyte like mTEC SPR, a Ciliated SPR, a Myelin SPR, a Myoid SPR, a Neuroendocrine SPR or a Tuft/lonocyte SPR. iTEC SPR values in Experiment 21/22 were close to 1 suggesting that the proportion of iTECs in this experiment is similar to the proportion of iTECs in the human thymus (the reference thymic map).

The cTEC Ratio and Relative cTEC Ratio (RCTR) were calculated for the samples described in Table 2 and results are presented in Table 4: As used herein, the cTEC ratio refers to the ratio of the proportion of cTEC high to cTEC low cells within a particular thymic map. RCTR refers to the ratio of cTEC Ratio in a test thymic map to a reference thymic map. RCTR values in Experiment 23 were close to 1 suggesting that the cTEC Ratio in this experiment is similar to the cTEC Ration in human thymus (the reference thymic map).

TABLE 3
SPRs for thymic cell derivations

| | | | | Exp 21 | Exp 21 | Exp 23 | Exp 23 |
|---|---|---|---|---|---|---|---|
| | Exp 7A | Exp 7B | Exp 7C | Rep 1 | Rep 2 | Rep 1 | Rep 2 |
| Aire+ mTEC-high SPR | 0.0 | 0.0 | 0.0 | 0.0 | 0.0 | 0.7 | 1.5 |
| Ciliated SPR | 1.0 | 1.4 | 0.7 | 1.4 | 2.1 | 0.1 | 0.1 |
| Corneocyte like mTEC SPR | 0.0 | 0.0 | 0.0 | 0.0 | 0.0 | 0.1 | 0.1 |
| cTEC-high SPR | 2.9 | 0.2 | 2.1 | 1.3 | 1.4 | 1.6 | 1.6 |
| cTEC-low SPR | 0.1 | 1.6 | 0.4 | 0.5 | 0.3 | 1.2 | 1.2 |
| iTEC SPR | 0.1 | 0.4 | 0.3 | 1.5 | 1.3 | 0.3 | 0.3 |
| mTEC-low SPR | 0.0 | 0.0 | 0.0 | 0.0 | 0.0 | 0.0 | 0.0 |
| Myelin SPR | 0.0 | 0.2 | 0.0 | 0.1 | 0.1 | 0.1 | 0.1 |
| Myoid SPR | 0.0 | 1.1 | 0.1 | 0.2 | 0.2 | 0.1 | 0.0 |
| Neuroendocrine SPR | 2.3 | 7.3 | 5.1 | 6.1 | 6.4 | 2.0 | 1.9 |
| Tuft/lonocyte SPR | 0.7 | 0.5 | 0.6 | 1.4 | 1.8 | 0.5 | 0.5 |

TABLE 4
RCTRs for thymic cell derivations

| | | Relative |
|---|---|---|
| Type | cTEC ratio | cTEC ratio |
| Human thymus | 0.86 | — |
| Exp 7A | 16.47 | 19.21 |
| Exp 7B | 0.09 | 0.10 |
| Exp 7C | 4.20 | 4.90 |
| Exp 21 Rep 1 | 2.44 | 2.85 |
| Exp 21 Rep 2 | 4.01 | 4.68 |
| Exp 23 Rep 1 | 1.12 | 1.31 |
| Exp 23 Rep 2 | 1.12 | 1.31 |

For Experiment 23 in particular, the analysis indicated that the iPSC derived thymic cells, from a transcriptional perspective, are predominantly similar to cTECs, iTECs, corneocyte-like mTECs and neuroendocrine cells.

UMAP and clustering analysis of the iPSC-derived thymic cells was performed. This analysis revealed the presence of 16 unbiased transcriptional clusters. Cluster-specific gene signatures were calculated using the algorithm CellRouter which identified the overlap of these signatures with gene expression signatures derived from the scRNA seq analysis of human thymus (described in Bautista et al. 2021). The heatmap shows the statistical significance of such enrichment. Cluster 16 specific genes were significantly enriched in the neuroendocrine gene signature from the human thymus, while cluster 11 possessed overlapping gene programs with corneocyte-like mTECs, and clusters 1, 4 and 5 showed significant signature overlap with cTECs. Taken together, these results indicated shared transcriptional programs between iPSC clusters and in vivo cell types in the human thymus. To identify putative cell types represented by each transcriptional cluster, the clustering and Symphony analyses were integrated. This analysis investigated how Symphony-predicted cell types are distributed across transcriptional clusters (Table 5).

TABLE 5
Proportion of different cell types in different clusters

| | Cluster No. (Across) | | | | | | | | | | | | | | | |
|---|---|---|---|---|---|---|---|---|---|---|---|---|---|---|---|---|
| Cell Type (Below) | 1 | 2 | 3 | 4 | 5 | 6 | 7 | 8 | 9 | 10 | 11 | 12 | 13 | 14 | 15 | 16 |
| Aire+ mTEC-high | 3 | 0 | 0 | 0 | 0 | 0 | 0 | 0 | 0 | 0 | 11 | 0 | 0 | 0 | 0 | 0 |
| Ciliated | 0 | 0 | 0 | 0 | 0 | 0 | 0 | 0 | 2 | 0 | 1 | 0 | 0 | 0 | 0 | 0 |
| Corneocyte-like mTEC | 0 | 0 | 0 | 0 | 0 | 0 | 0 | 0 | 0 | 0 | 10 | 0 | 0 | 0 | 0 | 0 |
| cTEC-high | 79 | 48 | 69 | 66 | 98 | 4 | 8 | 12 | 51 | 6 | 24 | 55 | 20 | 57 | 58 | 0 |
| cTEC-low | 3 | 39 | 22 | 21 | 2 | 93 | 75 | 82 | 2 | 78 | 29 | 22 | 68 | 39 | 4 | 1 |
| iTEC | 8 | 4 | 2 | 3 | 0 | 0 | 4 | 2 | 4 | 2 | 8 | 12 | 0 | 1 | 11 | 0 |
| mTEC-low | 0 | 0 | 0 | 0 | 0 | 0 | 0 | 0 | 0 | 0 | 1 | 0 | 0 | 0 | 0 | 0 |
| Myelin | 0 | 1 | 0 | 0 | 0 | 0 | 0 | 0 | 0 | 0 | 1 | 0 | 0 | 0 | 0 | 0 |
| Myoid | 0 | 1 | 0 | 0 | 0 | 0 | 0 | 0 | 0 | 0 | 1 | 1 | 0 | 0 | 0 | 0 |
| Neuroendocrine | 7 | 5 | 8 | 10 | 0 | 2 | 14 | 3 | 39 | 14 | 4 | 9 | 12 | 3 | 27 | 99 |
| Tuft/Ionocyte | 0 | 1 | 0 | 0 | 0 | 0 | 0 | 0 | 1 | 0 | 11 | 0 | 0 | 0 | 0 | 0 |

The majority of cells in cluster 16 were classified by Symphony as neuroendocrine cells, while cluster 1, 3, 4, 5 and 14 contained a high proportion of cells classified as cTECs, while cluster 6 contains mostly cells classified as cTEC-low. These data indicate that Symphony predictions can be used to guide identification of cell types from transcriptional clusters, which is a step often employed for exploratory data analysis.

To identify markers associated with neuroendocrine cells, cell markers expressed in cluster 16 were analyzed. Using CellRouter, the cell surface markers specifically expressed by cluster 16 in iPSC-derived thymic cells were analyzed. Cell surface markers for neuroendocrine cells included PDPN (ENSG00000162493), CNTN2 (ENSG00000184144), NCAM1 (ENSG00000149294), CXCR4 (ENSG00000121966), NGFR (ENSG00000064300), LiCAM (ENSG00000198910), CNTNAP2 (ENSG00000174469), ANK3 (ENSG00000151150), NTRK1 (ENSG00000198400), and/or SLC1A2 (ENSG00000110436). Using these cell surface markers, neuroendocrine cells may be removed from iPSC-derived thymic cell populations to generate cell populations enriched in other thymic cell types, such as, but not limited to, cTECs, and/or iTECs.

### Example 8: Overlapping Gene Expression Pattern Between iPSC-Derived Thymic Cells and Human Thymus

The gene expression programs potentially responsible for the molecular similarities between iPSC cells and cell types in the human thymus were investigated. From the heatmaps, overlapping gene sets between iPSC-derived thymic cell clusters and cell types in the human thymus were extracted and their mean expression levels were plotted onto the human thymus's expression levels. This analysis indicated that genes expressed in the iPSC cluster 1 and cTEC-high in the human thymus are indeed more highly expressed in this cell type relative to all others (see Table 6 for list of genes and associated ENSEMBL gene identifiers). Moreover, gene expression programs shared between cluster 11 and the corneocyte-like mTEC population are indeed more highly expressed in corneocyte-like TECs from the human thymus (see Table 7 for list of genes and associated ENSEMBL gene identifiers). Similarly, shared gene programs between cluster 16 and neuroendocrine cells are evident and, in this case, canonical neuroendocrine markers were identified in iPSC cells, such as NEUROD1, supporting the validity of our approach (see Table 8 for list of genes and associated ENSEMBL gene identifiers). Taken together, this analysis indicated the molecular similarities between iPSC-derived TEPs and their in vivo counterparts.

TABLE 6
Genes expressed in overlap of iPSC-thymic cell cluster 1
and cTEC-high group in human thymus

| Gene name | ENSEMBL Gene ID |
|---|---|
| PSMA3 | ENSG00000100567 |
| FABP5 | ENSG00000164687 |
| APRT | ENSG00000198931 |
| LSM6 | ENSG00000164167 |
| CTSV | ENSG00000136943 |
| SNRPE | ENSG00000182004 |
| ECHS1 | ENSG00000127884 |
| HSPE1 | ENSG00000115541 |
| RAN | ENSG00000132341 |
| TMA7 | ENSG00000232112 |
| TIMM13 | ENSG00000099800 |
| LDHB | ENSG00000111716 |
| ECI1 | ENSG00000167969 |
| GCSH | ENSG00000140905 |
| NOP58 | ENSG00000055044 |
| MRPL11 | ENSG00000174547 |
| STOML2 | ENSG00000165283 |
| ING2 | ENSG00000168556 |
| TOMM7 | ENSG00000196683 |
| MRPS34 | ENSG00000074071 |
| MRPL14 | ENSG00000180992 |
| MRPL57 | ENSG00000173141 |
| IMP3 | ENSG00000177971 |
| MZT2A | ENSG00000173272 |
| XRCC6 | ENSG00000196419 |

TABLE 7
Genes expressed in overlap of iPSC-thymic cell cluster 11
and corneocyte-like mTEC group in human thymus

| Gene name | ENSEMBL Gene ID |
|---|---|
| CD24 | ENSG00000272398 |
| ELF3 | ENSG00000163435 |
| CLDN4 | ENSG00000189143 |
| MAL2 | ENSG00000147676 |
| ASAH1 | ENSG00000104763 |
| TMEM123 | ENSG00000152558 |
| TMBIM6 | ENSG00000139644 |
| LGALS3 | ENSG00000131981 |
| MYL12B | ENSG00000118680 |
| ACADVL | ENSG00000072778 |
| KRT19 | ENSG00000171345 |
| SAT1 | ENSG00000130066 |
| RAB25 | ENSG00000132698 |
| WFDC2 | ENSG00000101443 |
| VAMP8 | ENSG00000118640 |
| SPINT1 | ENSG00000166145 |
| SERPINB1 | ENSG00000021355 |
| CDH1 | ENSG00000039068 |
| GSN | ENSG00000148180 |
| SDC4 | ENSG00000124145 |
| MGST2 | ENSG00000085871 |
| CAST | ENSG00000153113 |
| B4GALT1 | ENSG00000086062 |
| PERP | ENSG00000112378 |
| DMKN | ENSG00000161249 |

TABLE 8
Genes expressed in overlap of iPSC-thymic cell cluster
16 and neuroendocrine cell group in human thymus

| Gene name | ENSEMBL Gene ID |
|---|---|
| CPE | ENSG00000109472 |
| SOX4 | ENSG00000124766 |
| CDKN1C | ENSG00000273707; |
| | ENSG00000129757 |
| TUBB2A | ENSG00000137267 |
| STMN1 | ENSG00000117632 |
| MAP1B | ENSG00000131711 |
| TUBA1A | ENSG00000167552 |
| PCBP4 | ENSG00000090097 |
| GDI1 | ENSG00000203879 |
| SCG5 | ENSG00000277614; |
| | ENSG00000281931; |
| | ENSG00000166922 |
| RTN1 | ENSG00000139970 |
| ELAVL4 | ENSG00000162374 |
| NHLH1 | ENSG00000171786 |
| SOX11 | ENSG00000176887 |
| NEUROD1 | ENSG00000162992 |
| STMN2 | ENSG00000104435 |
| MLLT11 | ENSG00000213190 |
| PCSK1N | ENSG00000102109 |
| TUBB2B | ENSG00000137285 |
| CRMP1 | ENSG00000072832 |
| RPAIN | ENSG00000129197 |
| BASP1 | ENSG00000176788 |
| SSTR2 | ENSG00000180616 |
| CXCR4 | ENSG00000121966 |
| GAP43 | ENSG00000172020 |

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A thymic cell product generated by the process of:
a) generating one or more population of thymic cells;
b) extracting and sequencing RNA from each of the population of thymic cells;
c) analyzing RNA sequenced in a) to identify sub-populations within each population of thymic cells to generate a test thymic map for each of the population of thymic cells;
d) comparing each of the test thymic maps to a reference thymic map; and
e) identifying a thymic cell product by selecting a test thymic map that has a Sub-Population Ratio (SPR) or a Relative cTEC Ratio (RCTR) of from about 0.7 to 1.4.

2. The thymic cell product of claim 1, wherein the sub-populations within each population of thymic cells comprise one or more of immature thymic epithelial cells (iTECs), cTEC-high cells, cTEC-low cells, Aire+mTEC-high cells, mTEC-low cells, Corneocyte like mTEC cells, Ciliated cells, Myelin cells, Myoid cells, Neuroendocrine cells and Tuft/lonocyte cells.

3. The thymic cell product of claim 1, wherein the SPR of from about 0.7 to 1.4.

4. The thymic cell product of claim 3, wherein the SPR is a iTEC SPR, a cTEC-high SPR, a cTEC-low SPR, a Aire+mTEC-high SPR, a mTEC-low SPR, a Corneocyte like mTEC SPR, a Ciliated SPR, a Myelin SPR, a Myoid SPR, a Neuroendocrine SPR and a Tuft/lonocyte SPR.

5. The thymic cell product of claim 1, the iTEC SPR, the cTEC-high SPR, the cTEC-low SPR, the Aire+mTEC-high SPR, the mTEC-low SPR, the Corneocyte like mTEC SPR, the Ciliated SPR, the Myelin SPR, the Myoid SPR, the Neuroendocrine SPR and the Tuft/lonocyte SPR is from about 0.7 to 1.4.

6. The thymic cell product of claim 5, wherein the SPR is an the iTEC SPR, and wherein the iTEC is 1.

7. The thymic cell product of claim 1, wherein the RCTR is of from about 0.7 to 1.4.

8. (canceled)

9. The thymic cell product of claim 1, wherein the one or more populations of thymic cells are prepared by differentiation of stem cells.

10. (canceled)

11. The thymic cell product of claim 1, wherein the reference thymus map is prepared from one or more human thymi.

12. The thymic cell product of claim 1, wherein the human thymi are selected from fetal human thymi, postnatal human thymi, adolescent thymi, adult human thymi or a combination thereof.

13. The thymic cell product of claim 2, wherein the subpopulation comprises iTEC, and wherein iTEC comprises expression of one or more of IGKC (ENSG00000211592), ANAX2, LIMA1 (ENSG00000050405), EGFR (ENSG00000146648), ASCL1 (ENSG00000139352), HES1 (ENSG00000114315), JUND (ENSG00000130522), FOS (ENSG00000170345), ARID5B (ENSG00000150347), IRF1 (ENSG00000125347), MAFB (ENSG00000204103), IFI16 (ENSG00000163565), FOXC1 (ENSG00000054598), STAT1 (ENSG00000115415), JUNB (ENSG00000171223), EGFR1, ZFP36 (ENSG00000128016), JUN (ENSG00000177606), FOSB (ENSG00000125740), IER2 (ENSG00000160888), PAX9 (ENSG00000198807), and/or HIF1A (ENSG00000100644).

14. The thymic cell product of claim 2, wherein the subpopulation comprises cTEC, and wherein cTEC comprises expression of one or more of PSMA3 (ENSG00000100567), FABP5 (ENSG00000164687), APRT (ENSG00000198931), LSM6 (ENSG00000164167), CTSV (ENSG00000136943), SNRPE (ENSG00000182004), ECHS1 (ENSG00000127884), HSPE1 (ENSG00000115541), RAN (ENSG00000132341), TMA7 (ENSG00000232112), TIMM13 (ENSG00000099800), LDHB (ENSG00000111716), ECI1 (ENSG00000167969), GCSH (ENSG00000140905), NOP58 (ENSG00000055044), MRPL11 (ENSG00000174547), STOML2 (ENSG00000165283), ING2 (ENSG00000168556), TOMM7 (ENSG00000196683), MRPS34 (ENSG00000074071), MRPL14 (ENSG00000180992), MRPL57 (ENSG00000173141), IP3 (ENSG00000177971), MZT2A (ENSG00000173272), and/or XRCC6 (ENSG00000196419).

15. The thymic cell product of claim 2, wherein the subpopulation comprises corneocyte-like mTEC, and wherein corneocyte-like mTEC comprises expression of one or more of CD24 (ENSG00000272398), ELF3 (ENSG00000163435), CLDN4 (ENSG00000189143), MAL2 (ENSG00000147676), ASAH1 (ENSG00000104763), TMEM123 (ENSG00000152558), TMBIM6 (ENSG00000139644), LGALS3 (ENSG00000131981-), MYL12B (ENSG00000118680), ACADVL (ENSG00000072778), KRT19 (ENSG00000171345), SAT1 (ENSG00000130066), RAB25 (ENSG00000132698), WFDC2 (ENSG00000101443), VAMP8 (ENSG00000118640), SPINT1 (ENSG00000166145), SERPINB1 (ENSG00000021355), CDH1 (ENSG00000039068), GSN (ENSG00000148180), SDC4 (ENSG00000124145), MGST2 (ENSG00000085871), CAST (ENSG00000153113), B4GALT1 (ENSG00000086062), PERP (ENSG00000112378), and/or DMKN (ENSG00000161249).

16. A method for generating a population of thymic cells in vitro comprising:
culturing a population of cells in the presence of a soluble factor, a mineral or a combination thereof to induce differentiation or maturation of the population of cells to thymic cells;
wherein the population of cells is optionally engineered to express a cell surface receptor or an intracellular factor; and
wherein the population of cells comprises one or more cell types selected from the group consisting of pluripotent stem cells (PSCs), definitive endodermal (DE) cells, third pharyngeal pouch endodermal (PPE) cells, and anterior foregut endodermal (AFE) cells.

17. The method of claim 16, wherein the population of thymic cells comprises subpopulations of one or more of immature thymic epithelial cells (iTECs), cTEC-high cells, cTEC-low cells, Aire+mTEC-high cells, mTEC-low cells, Corneocyte like mTEC cells, Ciliated cells, Myelin cells, Myoid cells, Neuroendocrine cells and Tuft/lonocyte cells.

18-33. (canceled)

34. A method of maintaining a population of thymic cells in vitro comprising one or more of:
a) culturing the population of thymic cells in the presence of a soluble factor or a mineral;
b) culturing the population of thymic cells in the presence of one or more supporting cells; and/or
c) engineering the population of thymic cells to express a cell surface receptor or an intracellular factor.

35-45. (canceled)

46. The method of claim 34, wherein the population of thymic cells is engineered to express a cell surface receptor or an intracellular factor.

47-50. (canceled)

51. The method of claim 34, wherein the population of thymic cells is cultured in the presence of one or more supporting cells.

52. The method of claim 51, wherein the supporting cells are one or more of endothelial cells, mesenchymal stem cells, macrophages, dendritic cells (DCs), epithelial cells, fibroblasts, stromal cells, adipocytes, fibroblasts, vascular smooth muscle cells (VSMCs), or lymphatic endothelial cells.

53-54. (canceled)

55. The method of claim 34, wherein the population of thymic cells comprises sub populations of one or more selected from the group consisting of thymic epithelial progenitor cell (TEPCs), immature thymic epithelial cells (iTECs), thymic epithelial cells (TECs), medullary thymic epithelial cells (mTECs) and cortical thymic epithelial cells (cTECs).
